(19) United States
(12) Patent Application Publication
(10) Pub. No.: US 2022/0330558 A1
(43) Pub. Date: Oct. 20, 2022

(54) FUNCTIONAL FOOD

(71) Applicants: Roberto Battoia, Udine (IT); Maria Jose' Martinez Del Valle, Valencia, Valencia (ES)

(72) Inventors: Roberto Battoia, Udine (IT); Maria Jose' Martinez Del Valle, Valencia, Valencia (ES)

(21) Appl. No.: 17/621,248

(22) PCT Filed: Jun. 24, 2020
(86) PCT No.: PCT/IB2020/055958

§ 371 Date: Dec. 21, 2021

(30) Foreign Application Priority Data
Jun. 26, 2019 (IT) 102019000010110

(51) Int. Cl. A21D 2/38; A23L 33/105; A23L 33/22; A21D 10/00; A23L 7/20; A61K 31/201; A61K 31/202; A61K 31/01; A61K 31/7048; A61K 31/05; A61K 31/192; A61P 3/10

(52) U.S. Cl. CPC A21D 2/38; A23L 33/105; A23L 33/22; A21D 10/005; A23L 7/20; A61K 31/201; A61K 31/202; A61K 31/01; A61K 31/7048; A61K 31/05; A61K 31/192; A61P 3/10; A23V 2002/00

(57) ABSTRACT

It is disclosed a food mixture, in particular a food mixture comprising hydroxytyrosol, oleuropein, and soluble fibres. In particular, the present invention relates to a food mixture comprising: —a fraction (a) which comprises soluble fibres; —a powdered fraction (b) which comprises at least 100 mg/kg hydroxytyrosol and at least 250 mg/kg oleuropein where said amounts refer to said powdered fraction (b). Said food mixture finds application, moreover, in the preparation of bread, pasta, and baked goods in general. The invention also describes food products which contain this mixture, a process for the preparation thereof, as well as the uses thereof in human and animal nutrition, in the treatment and in the prevention of inflammatory conditions and in the reduction of postprandial glycaemia.

### FIELD OF THE INVENTION

The present invention relates to a food mixture comprising hydroxytyrosol, oleuropein, and soluble fibres, the process for its preparation, as well as uses thereof in human and animal nutrition, in the treatment and in the prevention of inflammatory states and in the reduction of the postprandial glycaemia.

### BACKGROUND ART

The Mediterranean diet is characterised by a high consumption of products derived from cereals, such as pasta, bread and, more generally, baked goods. Containing a large amount of carbohydrates, these products usually have a high glycaemic index, i.e. cause a high rise in the post-prandial glycaemia.

Post-prandial hyperglycaemic phenomena can harm people with manifest diabetes or with low glucose tolerance. For example, a relationship has been found between postprandial hyperglycaemia and the development of the diabetes-related complications, such as degenerative diseases affecting the eyes, kidneys, cardiovascular system, and nervous system. For this reason, achieving optimal glycaemic control is taking on ever greater importance in the treatment of diabetes, with a view to reducing the postprandial glycaemic excursions, through—for example—the adoption of a ketogenic or low-carb diet.

These types of diet are however utilised also by people not suffering from diabetes or low glucose tolerance, since the low intake of carbohydrates with the diet forces the body to produce ketone bodies and the body and the brain, in particular, utilise the ketone bodies as a source of energy, with consequent increase in the energy consumption of fats contained in fat tissue. Originally developed for the treatment of drug-resistant epilepsy, ketogenic diets are largely used today to promote weight loss and, more recently, in the field of sports nutrition.

Furthermore, ketone bodies have a high anti-inflammatory potential, and the ketogenic diets are suitable to be utilised as coadjutant preventive and/or protective nutritional treatments for the neurological and rheumatic illnesses.

Unfortunately, in the medium/long term, it has been recorded that a very low percentage of people maintain these types of diet, as they require considerable effort in terms of concentration, discipline, and sacrifice.

In this context, the need is felt in the sector for foods which combine a low glycaemic index and anti-inflammatory activity, which can therefore be utilised not only in the context of a ketogenic diet but also of a Mediterranean diet, which is much easier to maintain and with which the possibilities of success are therefore better.

### SUMMARY OF THE INVENTION

Said object has been achieved by a food mixture, in particular a food mixture for use in the preparation of food products such as pasta or baked goods, comprising:
a fraction (a) comprising soluble fibres;
a powdered fraction (b) comprising at least 100 mg/kg hydroxytyrosol and at least 250 mg/kg oleuropein, where said amounts refer to said powdered fraction (b).

In another aspect, the invention relates to a food product comprising the food mixture, where said product is selected from: a flour or mixture of flours and derived products thereof (such as baked goods and pasta), fruit juices, fruit marmalades and jams, ice-creams, beverages, and beers.

In a further aspect, the invention relates to the food mixture or food product for use in the reduction of postprandial glycaemia in healthy mammals, and/or in the treatment and/or in the prevention of inflammation.

For the objects of the present invention, the term “inflammation” means any pathological state or condition associated with inflammatory colorectal diseases, in particular Crohn's disease, irritable bowel syndrome, and colitis.

### DESCRIPTION OF THE DRAWINGS

The characteristics and advantages of the present invention will become apparent from the following detailed description and the working examples provided for illustrative and non-limiting purposes, as well as the accompanying figures, wherein:

FIG. 1: Variation of glycaemia (mg/dl) in the time after the consumption of the foods stated. Dashed line=glucose, dotted line=white bread, continuous line=bread comprising the food mixture according to the present invention.

FIG. 2: levels of IL6 in the serum before and after consumption of bread comprising the mixture according to the present invention for 5 consecutive days.

### DETAILED DESCRIPTION OF THE INVENTION

In the context of the present description and in the appended claims, the term ‘(pseudo)cereals’ means herbaceous plants that produce fruit which can be ground into flour for making pasta or baked goods. These (pseudo)cereals can be monocotyledon plants belonging to of the Poaceae family (e.g. corn) and plants not belonging to this family (e.g. belonging to the Polygonacee family).

In the context of the present description and of the appended claims, the term ‘baked goods’ means goods including bread, pastries, and biscuits.

The term ‘soluble fibres’ means those fibre selected from the group consisting of low-molecular-weight polysaccharides, oligosaccharides, galactooligosaccharides, fructooligosaccharides, beta-glucans, pectins, inulin, resistant starch, mucilage, and mixtures thereof, principally fibre constituted of polysaccharide chains of arabinoxylans belonging to family of the pentosans and ferulic acid, an antioxidant molecule.

In a first aspect, the present invention therefore relates to a food mixture, preferably but not exclusively, for use in the preparation of food products such as pasta or baked goods, comprising:
a fraction (a) comprising soluble fibres;
a powdered fraction (b) comprising at least 100 mg/kg hydroxytyrosol and at least 250 mg/kg oleuropein, where said amounts refer to said powdered fraction (b).

In a preferred embodiment, said fraction (a) comprises up to 30 mg beta-glucans and up to 300 mg pentosans with a molecular weight not higher than 30 kDa per gram of said fraction (a) in dried form.

Still more preferably, said fraction (a) comprises up to 25 mg beta-glucans and up to 250 mg pentosans with a molecular weight not higher than 30 kDa per gram of said fraction (a) in dried form.

In a preferred embodiment, said aqueous fraction further comprises ferulic acid, preferably up to 0.5 mg per gram of said fraction (a) in dried form.

In a preferred embodiment, said fraction (a) is obtained from brewers' spent grain, i.e. a by-product of the brewing industry, where the term “brewers' spent grain” means the residue of the hot extraction of malted cereal: they comprise the husk of the grain and the fractions which not have undergone solubilisation during the malting and mashing process, as well as variable amounts of starch and of dextrins which have not been saccharified.

In a particularly preferred embodiment, the fraction (a) is a brewers' spent grain hydrolysate, more preferably is a brewers' spent grain hydrolysate obtained by hydrolysis in the presence of xylanase.

Preferably, said fraction (a) is obtained in accordance with the process described in the Italian patent application n. 102019000005588, said process comprising the following steps:
i) mixing brewers' spent grain and water, in a weight ratio of 1:1 to 1:5,
ii) adding to the mixture thus obtained up to 2 wt %, based on the mixture weight, an enzyme consisting of xylanase, and leaving to react for 1-6 hours at a temperature of 45-65° C. and at a pH of 4-6,
iii) deactivating the enzyme from step ii), by increasing the temperature to 80-90° C. for at least 5 minutes, and
iv) separating the liquid component from the solid component obtained at the end of step iii), and keeping the liquid component, i.e. the brewers' spent grain hydrolysate.

Preferably, said xylanase is endo-1,4-beta-xylanase.

Preferably, in step i), brewers' spent grain and water are in a weight ratio of 1:1.5 to 1:3.

Preferably, in step ii), the enzyme is added in an amount up to 1 wt %, based on the mixture weight. In more preferred embodiments, in step ii), the enzyme is added in an amount of 0.1-0.7 wt %, based on the mixture weight.

Preferably, in step ii) the mixture is left to react for 2-4 hours at 58-62° C.

In step iii), the enzyme is deactivated by a thermal treatment, i.e. by raising the temperature. Preferably, the deactivation takes place at approximately 85° C. for approximately 15 minutes.

In step iv), the separation of the liquid component can be carried out by known filtration or/and centrifugation techniques. Optionally, further washes with water are also possible.

Optionally, the process of preparation further comprises a step v) for the desiccation of the liquid component derived from step iv), obtaining, in that way, a dried brewers' spent grain hydrolysate. Step v), for the desiccation, is preferably carried out at temperatures no higher than 65° C. This step v), for the desiccation, can be carried out by means of lyophilisation.

From the process set out above, therefore a brewers' spent grain hydrolysate is obtained which comprises ferulic acid, proteins, up to 30 mg beta-glucans and up to 300 mg pentosans with a molecular weight not higher than 30 kDa per gram of dried hydrolysate. The expression ‘desiccated hydrolysate’ or ‘dried hydrolysate’ means a hydrolysate which has undergone desiccation. A hydrolysate is deemed desiccated or dried if it features a residual water content not higher than 1 wt %, based on the weight of the dried hydrolysate.

Preferably, the brewers' spent grain hydrolysate comprises up to 25 mg beta-glucans and up to 250 mg pentosans with a molecular weight of not higher than 30 kDa per gram of dried hydrolysate.

Preferably, the brewers' spent grain hydrolysate comprises up to 0.5 mg ferulic acid per gram of dried hydrolysate.

Preferably, the brewers' spent grain hydrolysate comprises up to 8 mg proteins per gram of dried hydrolysate.

In some preferred embodiments, the brewers' spent grain hydrolysate is a dried hydrolysate. Having a dried product offers a series of advantages which range from the handleability of low volumes compared to corresponding aqueous solutions, to the consequent ease of transportation, in addition to the storage times, due to the significant reduction of the risk of both bacterial contamination and rancidification. Naturally, should the circumstances required it, the hydrolysate could easily be solubilised in water to bring the product in liquid form to the desired concentration.

The fraction (a) comprising soluble fibres can therefore be liquid or solid, in particular powdered. In one embodiment, the fraction (a) can be a liquid or dried brewers' spent grain hydrolysate. In a preferred embodiment, the fraction (a) comprising soluble fibres is a powder, preferably a dried brewers' spent grain hydrolysate.

Preferred embodiments are those wherein, in the food mixture according to the invention, the fraction (a) comprises a brewers' spent grain hydrolysate comprising ferulic acid, proteins, up to 30 mg beta-glucans, and up to 300 mg pentosans with a molecular weight not higher than 30 kDa per gram of dried hydrolysate. More preferred embodiments are those wherein this hydrolysate is obtained by the process comprising steps i)-iv) described above.

The determination of the pentosans is based on a colorimetric method which is rapid and reproducible and based on phloroglucinol (S. G. Douglas, “A rapid method for the determination of pentosans in wheatflour”, Food Chemistry, 7, 1981, 139-145) using D-xylose at 510-552 nm as a reference standard. Since the method envisages a high-temperature acid hydrolysis, all the pentosans are counted regardless of the degree of polymerisation thereof. For the purposes of the present invention, the number average molecular weight (Mn) of the pentosans in the hydrolysate is measured by means of diafiltration through sieves with different molecular cut-offs: 50 kDa, 30 kDa, 10 kDa and 5 kDa.

The ferulic acid is preferably quantified by reverse stage HPLC analysis on column C18 utilising a gradient of acetonitrile in water and trifluoroacetic acid.

Since said ferulic acid is present in a form which is released from by the structures of the plant fibre, it is more bioavailable and therefore more effective.

In some embodiments, said brewers' spent grain hydrolysate consists essentially of ferulic acid, proteins, up to 30 mg beta-glucans, and up to 300 mg pentosans with a molecular weight not higher than 30 kDa per gram of dried hydrolysate. The expression “consists essentially of” means that ferulic acid, proteins, beta-glucans, and pentosans with a molecular weight not higher than 30 kDa are the sole active ingredients present in the hydrolysate, while any further components or excipients do not interfere with the action thereof. It should be understood that all the aspects specified above as preferred and advantageous for the hydrolysate and the components thereof, must likewise be deemed to be preferred and advantageous for these embodiments.

In other embodiments, said brewers' spent grain hydrolysate consists of ferulic acid, proteins, up to 30 mg beta-glucans and up to 300 mg pentosans with a molecular weight not higher than 30 kDa per gram of dried hydrolysate, and optionally water.

The hydrolysate as described above is particularly advantageous for the purposes of the present invention. Indeed, it is advisable to consider the following.

The pentosans, and in particular the arabinoxylans, regulate the adsorption of sugars and fats contributing to the control of the level of glucose and cholesterol in the blood. They play therefore an active role in the reduction of glycaemia and in controlling hypercholesterolemia and obesity. Furthermore, arabinoxylans are well known to be prebiotics which can increase faecal bifidobacteria and reduce urinary excretion of p-cresol, leading to an overall improvement of intestinal health and the immune system.

However, the large majority of the pentosans, including arabinoxylans, and the ferulic acid present in the food-grade plant fibre in general, is not bioavailable since strictly connected to other inert structures, such as cellulose and lignin.

The very incapacity of the digestive tract to break down these structures leads the probiotic microflora in the large intestine to produce hydrolytic enzymes which, in the presence of food-grade fibre, separate pentosans and ferulic acid from the compounds to which they are connected, making them bioavailable. However, this process is slow and, since only occurring in the end section of the intestinal tract, has very low yields.

While the positive effects of the consumption of plant fibres are certain, it must be noted that consumption of 8 grams of arabinoxylan-rich fibre for every 100 grams of carbohydrates can lead to a series of disturbances, such as—for example—bloating, meteorism, colon irritation, and consequent abdominal pain, due to the portion of insoluble fibres.

Furthermore, the whole foods which must be consumed in order to reach a content of arabinoxylan-rich fibre amounting to 8 g out of 100 g, are in general less appetising and contain lipase inhibitors, which render ineffective the pancreatic lipases, and phytates, which cannot be digested by humans or non-ruminant animals and are classified as anti-nutritional due to the chelating effects thereof on some nutritional elements. Indeed, if consumed in large amounts, phytates inhibit the metabolism and the absorption of numerous minerals, such as calcium and zinc, in addition to vitamin B1, rendering certain proteins indigestible.

A further consideration which must be made is that the insoluble part of the food-grade plant fibre may be contaminated with mycotoxins, which alter the immune and neurological systems, cause oxidative stress and harm the intestinal barrier.

The hydrolysate as described above is therefore particularly advantageous as obtained by a process which has made it into a product with high bioavailability of soluble fibres, which allow, at the same time, to reduce the advantages associated with a high consumption of commonly known wholegrain products. Furthermore, it reutilizes a brewery waste product, obtaining materials which find various applications and eliminate overall waste.

In an embodiment, said powdered fraction (b) further comprises 45 mg/kg or less of at least one natural triterpene and/or 10 mg/kg or less of squalene.

In a preferred embodiment, said powdered fraction (b) comprises at least 110 mg/kg hydroxytyrosol, or 119 mg/kg thereof, and at least 260 mg/kg oleuropein, or 264 mg/kg thereof, where said amounts refer to said powdered fraction.

In a preferred embodiment, said powdered fraction (b) further comprises 500 mg/kg or less of omega-3 and/or omega-6 fatty acids, preferably approximately 200-500 mg/kg, and even more preferably approximately 300-400 mg/kg.

In a preferred embodiment, the fatty acids can be omega-3 fatty acids, more preferably they may be selected from between eicosapentaenoic acid (EPA) and/or docosahexaenoic acid (DHA).

In a preferred embodiment, said powdered fraction (b) further comprises powder obtained from grinding olive stone, still more preferably by grinding the seeds contained inside olive stones. Said powder can be obtained from the process described in patent application ES2389820, here recalled in its entirety. According to this process, the whole olive stones obtained from any stone removal system are broken without damaging the seed and subsequently the seeds are separated mechanically from the shells. The seeds are desiccated to eliminate the residual humidity and then cold-pressed, preferably in a continuous press. The cake obtained from the press is ground and micronized to obtain a powder with the desired characteristics, preferably with a particle size of 500 μm or less.

The powder thus obtained from the seeds of olive stones, comprises proteins, fats, water, fibre, carbohydrates, triterpenes, and squalene.

In particular, this powder comprises 200-300 mg/kg maslinic acid, 100-200 mg/kg oleanolic acid, and 50-100 mg/kg squalene, where “mg/kg” means mg per kg of powder.

The powder furthermore comprises several free amino acids, such as aspartic acid, histidine, arginine, alanine, proline, tyrosine, valine, methionine, lysine, isoleucine, leucine, phenylalanine, glutamine, asparagine, and tryptophan.

It is therefore a powder also incorporating significant amounts of oleanolic acid, maslinic acid, and squalene, which make it a plant-based flour which can be very competitive as an ingredient or additive in the processing of functional foods.

Therefore, alternatively, the food mixture according to the invention comprises:
a fraction (a) comprising soluble fibres;
a powdered fraction (b) which further comprises powder obtained from the grinding of the seeds contained in the olive stones and which comprises 45 mg/kg or less of at least one natural triterpene and/or 10 mg/kg or less of squalene, where said amounts refer to said powdered fraction (b).

Preferably, said at least one natural triterpene is oleanolic acid, maslinic acid, or a combination thereof.

While the powder obtained from the grinding of the seeds contained in the olive stones offers all the nutritional properties stated above, the said powder has the disadvantage of being characterised by a very intense bitter flavour, which has so far discouraged its use in food products. In this regard, it has surprisingly been found that when this powder is mixed with the brewers' spent grain hydrolysate, as described above, the resulting food mixture offers a decidedly more pleasant flavour, thereby advantageously making possible to exploit both the characteristics of the powder obtained from the grinding of the seeds contained in the olive stones and the characteristics of the brewers' spent grain hydrolysate. Indeed, unexpectedly, the brewers' spent grain hydrolysate suitably masks the bitter flavour of the powder, guaranteeing the resulting mixture a pleasant flavour and good palatability. Without wishing to be bound by any theory, it is believed that this effect of masking bitter flavour is due to the presence of sugars, such as xylose, in the said hydrolysate.

Embodiments which are more preferred are therefore those wherein the food mixture according to the invention comprises:
a fraction (a) comprising a brewers' spent grain hydrolysate comprising ferulic acid, proteins, up to 30 mg beta-glucans, and up to 300 mg pentosans with a molecular weight not higher than 30 kDa per gram of dried hydrolysate; and
a powdered fraction (b) which, in addition to hydroxytyrosol and oleuropein, further comprises powder obtained from the grinding of the seeds contained in the olive stones and comprises 45 mg/kg or less of at least one natural triterpene and/or a 10 mg/kg or less of squalene, where said amounts refer to said powdered fraction (b).

It should be understood that all the aspects specified above as preferred and advantageous for the brewers' spent grain hydrolysate and powder from seeds of olive stones, should be likewise deemed to be preferred and advantageous for these embodiments comprising both.

Preferably, in the food mixture, the amounts of powder obtained from the grinding of the seeds contained in the olive stones is greater than the amounts of brewers' spent grain hydrolysate.

More preferably, said powder from olive stones and said brewers' spent grain hydrolysate are in a weight ratio of 2:1 to 5:1.

In particularly preferred embodiments, said powder from olive stones and said brewers' spent grain hydrolysate are in a weight ratio of 3:1.

Preferably, the food mixture comprises all the components stated above.

The food mixture comprises preferably 10-30 wt % of the fraction (a) and 70-90 wt % of the powdered fraction (b), where the amounts of (a) and (b) refer to the weight of the food mixture.

In a further aspect, the present invention relates to food products comprising the composition according to the present invention. By way of a non-limiting example, said food products are selected from a flour or mixture of flours and derived products thereof (such as baked goods and pasta), fruit juices, fruit marmalades and jams, ice-creams, beverages, and beers.

In another aspect, the present invention relates to a food-grade flour made of at least one (pseudo)cereal comprising a 15 wt % or less of the food mixture as described above in detail, preferably 3-10 wt %, and more preferably 5-7 wt %, wherein the said amounts refers to the overall weight of the food-grade flour comprising the food mixture.

When the food mixture is in the preferred embodiments wherein it comprises said brewers' spent grain hydrolysate, said food-grade flour preferably comprises up to 5 wt % of hydrolysate, in relation to the weight of the food-grade flour.

When the food mixture is in the preferred embodiments wherein it comprises said powder from olive stones, said food-grade flour preferably comprises up to 15 wt % of powder from olive stones, in relation to the weight of the food-grade flour.

When the food mixture is in the preferred embodiments wherein it comprises said brewers' spent grain hydrolysate and said powder from olive stones, said food-grade flour preferably comprises up to 5 wt % of hydrolysate and up to 15 wt % of powder from olive stones, in relation to the weight of the food-grade flour.

More preferably, said food-grade flour preferably comprises 0.1-3 wt % of hydrolysate and 1-8 wt % of powder from olive stones, in relation to the weight of the food-grade flour.

In particularly preferred embodiments, said food-grade flour comprises 0.5-2 wt % of hydrolysate and 1.5-6 wt % of powder from olive stones, in relation to the weight of the food-grade flour.

In the more preferred embodiments, said food-grade flour comprises 1 wt % of hydrolysate and 3 wt % of powder from olive stones, in relation to the weight of the food-grade flour.

Preferably, the (pseudo)cereal can be selected from the group consisting of wheat, rye, buckwheat, barley, oats, corn, rice, and combinations thereof.

The food-grade flour can be any of the following Italian flours types: type 2, type 1, type 0, type 00 and combinations thereof, preferably it is at type 0 or type 00 flour, depending on the type of product which will be made.

In a further aspect, the present invention relates to the food mixture and the food products comprising the same for use in the reduction of glycaemia, in particular of the postprandial glycaemia in healthy mammals, and/or for use in the prevention and/or in the treatment of inflammation, in particular in the prevention and/or in the treatment of inflammation associated with inflammatory colorectal diseases, preferably Crohn's disease, irritable bowel syndrome, and colitis.

In order to demonstrate the significant efficacy of the food mixture according to the invention in the reduction of postprandial glycaemia and on inflammation, the following examples show the tests performed on healthy people, i.e. people not suffering from full-blown diseases and evident inflammatory conditions. This choice allowed to demonstrate that the efficacy is observed even under normal health conditions, thus confirming the preventive action of the onset of any pathological state. At the same time, it follows that, in the treatment of patients with chronic inflammatory conditions, such as for example people suffering from obesity or diabetes or from chronic diseases, the effect will consequently be much more significant, i.e. an actual and true therapeutic effect will be observed.

It should be understood that all the possible combinations of the preferred aspects of the food mixture, the individual fractions (a) and (b), its preparation process and uses and the products containing them, as stated above are to be deemed as hereby disclosed, and likewise preferred.

It should also be understood that all the aspects specified as preferred and advantageous for the food mixture and the fractions (a) and (b) thereof, should therefore be deemed to be likewise preferred and advantageous also for the preparation and the uses thereof.

Below are working examples of the present invention provided for illustrative purposes.

### EXAMPLES

The following analytical methods have been utilised to determine the properties stated in the description and in the examples.

Determination of the pentosans: the pentosans have been determined applying a phloroglucinol-based colorimetric method which is rapid and reproducible (S. G. Douglas, “A rapid method for the determination of pentosans in wheatflour”, Food Chemistry, 7, 1981, 139-145) using D-xylose at 510-552 nm as a reference standard. Since the method envisages a high-temperature acid hydrolysis, all the pentosans are counted regardless of the degree of polymerisation thereof. For the purposes of the present invention, the number average molecular weight (Mn) of the pentosans in the hydrolysate is measured by means of diafiltration through sieves with different molecular cut-offs: 50 kDa, 30 kDa, 10 kDa and 5 kDa.

### Example 1

Preparation of Fraction (a)

A brewers' spent grains hydrolysate was prepared according to the present invention, by setting the following process parameters:

i) brewers' spent grains and water were mixed, wherein the brewers' spent grains being 40 wt %,

ii) the enzyme was added in an amount of 0.2 wt %, at a pH of 5, by raising the temperature to 60° C., for 4 hours,

iii) the enzyme was then deactivated by heating to 85° C. for 15 minutes, iv) the resulting hydrolysate was then separated from the remaining solids by filtration.

Results:

| | | Example 1 |
|---|---|---|
| | | endo-1,4- |
| | | beta-xylanase |
| TOTAL SOL. PENTOSANS | phloroglucinol | 4.417 |
| [mg/ml] of cui: | | |
| XYLOSE [mg/ml] | enzymatic kit | 0.205 |
| ARABINOSE [mg/ml] | enzymatic kit | 0.015 |
| FREE FERULIC ACID [ppm] | HPLC | 1.47 |
| PROTEINS [ppm] | Bradford | 63.8 |
| BETA-GLUCANS [ppm] | enzymatic kit | 245.6 |
| DRIED RESIDUE [%] | gravimetric | 1.41 |
| WATER CONTENT [%] | by difference | 98.59 |

After a subsequent step v) of desiccation, the hydrolysate had the following composition:

| | | Example 1 |
|---|---|---|
| | | endo-1,4- |
| | | beta-xylanase |
| TOTAL SOL. PENTOSANS | phloroglucinol | 313.26 |
| [mg/g] of cui: | | |
| XYLOSE [mg/g] | enzymatic kit | 14.54 |
| ARABINOSE [mg/g] | enzymatic kit | 1.06 |
| FREE FERULIC ACID [mg/g] | HPLC | 0.104 |
| PROTEINS [mg/g] | Bradford | 4.524 |
| BETA-GLUCANS [mg/g] | enzymatic kit | 17.418 |
| DRIED RESIDUE [%] | gravimetric | 100.00 |
| WATER CONTENT [%] | by difference | — |

Distribution of the molecular weights of the arabinoxylans obtained also in dried hydrolysate:

The analysis of the distribution by filtration through a cut-off membrane is as follows:

| Pentosans: | Example 1 |
|---|---|
| 10-30 kDa | 17% |
| 5-10 kDa | 29% |
| <5 kDa | 13% |

### Example 2

Evaluation of the Anti-Inflammatory Activity and the Glycaemic Index of Bread Comprising the Food Mixture According to the Invention

Six people were enrolled, i.e. 3 men and 3 women.

The glycaemic index was assessed by using the a Glucomen Menarini Diagnostic device and a taking a capillary blood sample from a fingertip.

Each subject consumed (at three different times): 50 g glucose, 70 g conventional white bread, and 80 g of bread made with a flour comprising 15 wt % of the food mixture according to the present invention, in which the food mixture contained 10 wt % of powdered fraction (b) containing 119.12 mg/kg hydroxytyrosol and 263.98 mg/kg oleuropein and 90 wt % of fraction (a) consisting of a dried brewers' spent grain hydrolysate, as stated above and prepared in Example 1.

The amounts were calibrated so as to have the same amount of carbohydrates in each administration.

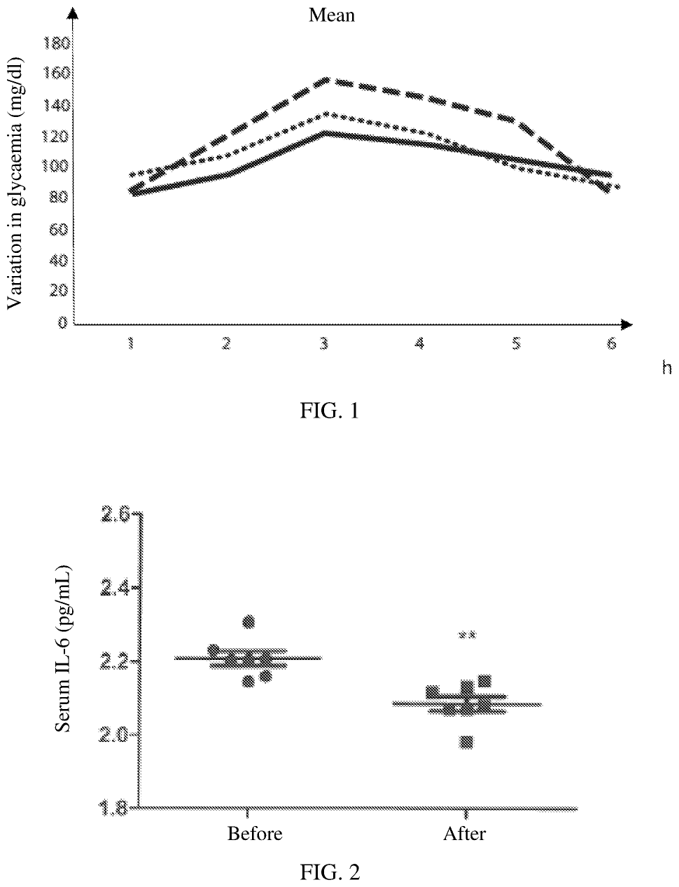

Blood samples were taken before the food was administered and then after 30, 45, 60, 90, and 120 minutes. FIG. 1 shows the graph of the variation in glycaemia experienced by the subjects over time. The glycaemic response and, therefore, the glycaemic index were lower over time following administration of bread comprising the mixture according to the present invention.

The glycaemic index calculated by applying the ratio between the area under the curve of the glucose and the area under the curve of the bread with the food mixture according to the present invention is approximately 46, which places it in a low glycaemic index (below GI55) zone. It is significant to note that the same type of use of a bread product with the same flour but without the mixture according to the present invention, leads to a glycaemic index o approximately 76, which is therefore much higher than that observed with the bread comprising the food mixture according to the present invention. On a different day of the three tests, but with the same people, a venous blood sample was taken. The same people, without changing anything else in their food habits or lifestyle, except the daily consumption for 5 days in a row of 100 g bread comprising the mixture according to the present invention, underwent further venous blood samples after said 5 days.

Interleukin 6 (1L6), an inflammatory cytokine, was then assessed using an ELISA test. The data is shown in FIG. 2. The reduction in the levels of IL6 after 5 days of consumption of the bread comprising the food mixture according to the present invention was statistically significant.

The data demonstrates the anti-inflammatory action of the bread comprising the mixture according to the present invention. The result should be deemed particularly significant where obtained in healthy people, who do not have full-blown diseases or inflammatory conditions, therefore the action is efficacious in decreasing inflammation due to normal everyday life. The impact is much greater in people who have chronic inflammatory conditions, such as for example people suffering from obesity or diabetes or from chronic diseases.

1. A food mixture for preparing food products said food mixture comprising:
a fraction (a) comprising soluble fibres;
a powdered fraction (b) comprising at least 100 mg/kg hydroxytyrosol, and at least 250 mg/kg oleuropein, wherein said amounts refer to said powdered fraction (b).

2. The food mixture according to claim 1, where the fraction (a) comprises up to 30 mg beta-glucans and up to 300 mg pentosans having a number average molecular weight not higher than 30 kDa per gram of said fraction (a) in dried form.

3. The food mixture according to claim 1, wherein the fraction (a) further comprises ferulic acid, preferably up to 0.5 mg thereof per gram of said fraction (a) in dried form.

4. The food mixture according to claim 1, where the fraction (a) is obtained by brewers' spent grain.

5. The food mixture according to claim 1, wherein the fraction (a) is in the form of powder, and is a dried brewers' spent grainhydrolysate.

6. The food mixture according to claim 1, wherein the powdered fraction (b) comprises 45 mg/kg or less of at least one natural triterpene and/or 10 mg/kg or less of squalene.

7. The food mixture according to claim 1, wherein the powdered fraction (b) further comprises 1000 mg/kg or less of omega-3 and/or omega-6 fatty acids.

8. The food mixture according to claim 1, wherein the powdered fraction (b) is obtained by grinding olive stones.

9. The food mixture according to claim 1 comprising 10-30 wt % of fraction (a) and 70-90 wt % of powdered fraction (b), wherein the amounts of (a) and (b) refer to the weight of the food mixture.

10. A food product comprising the food mixture according to claim 1, wherein said food product is selected from: a flour or mixture of flours, baked goods, pasta, fruit juices, fruit marmalades and jams, ice-creams, beverages, and beers.

11. The food product according to the claim 10, wherein the product is a food-grade flour or a mixture of food-grade flours comprising 15 wt % or less of the food mixture according to claim 1.

12. A method for reducing postprandial glycaemia in healthy mammals, and/or treating and/or preventing inflammation, said method comprising administering an effective amount of the food mixture according to claim 1 to a subject in need thereof.

13. The method according to claim 12, wherein inflammation is associated with colorectal inflammatory diseases comprising Crohn's disease, irritable bowel syndrome, or colitis.
